(19) United States
(12) Patent Application Publication
(10) Pub. No.: US 2019/0247224 A1
(43) Pub. Date: Aug. 15, 2019

(54) Personal Heating Dexterity Device

(71) Applicant: THE UNITED STATES OF AMERICA AS REPRESENTED BY THE SECRETARY OF THE ARMY, FORT DETRICK, MD (US)

(72) Inventors: John CASTELLANI, Oxford, MA (US); Timothy DRISCOLL, Oviedo, FL (US)

(73) Assignee: THE UNITED STATES OF AMERICA AS REPRESENTED BY THE SECRETARY OF THE ARMY, FORT DETRICK, MD (US)

(21) Appl. No.: 16/333,965

(22) PCT Filed: Feb. 14, 2018
(86) PCT No.: PCT/US2018/018105

§ 371 Date: Mar. 15, 2019

Related U.S. Application Data
(60) Provisional application No. 62/459,131, filed on Feb. 15, 2017.

(51) Int. Cl. A61F 7/02; A61F 7/00; A41D 13/005

(52) U.S. Cl. CPC A61F 7/02; A61F 7/007; A41D 13/0051; A61F 2007/0007; A61F 2007/0233; A61F 2007/0034; A61F 2007/0086; A61F 2007/0255; A61F 2007/0282; A61F 2007/0015

(57) ABSTRACT

An apparatus for improving dexterity of the bare hands in cold temperatures includes heating pads 10 disposed on the forehead, cheeks and forearms. The heating pads 10a, 10b, 10c on the forehead and cheeks stimulate the trigeminal nerve area to increase blood flow to the fingers. The heating pads 10d, 10d on the forearms 24, 24 increase the temperature of the blood flowing to the hands. Thermocouples 32 in each heating pad 10 are connected to a set-point controller 28.

### CROSS REFERENCE TO RELATED APPLICATIONS

The present application claims the benefit of priority of U.S. provisional patent application Ser. No. 62/459,131 filed on Feb. 15, 2017, which is expressly incorporated by reference herein.

### STATEMENT OF GOVERNMENT INTEREST

The invention described herein may be manufactured, used and licensed by or for the United States Government.

### BACKGROUND OF THE INVENTION

The invention relates in general to personal heating devices and in particular to personal heating devices for maintaining dexterity of the hands.

Cold-weather operations pose unique problems with regard to maintaining comfort, injury protection and performance. Many military operators list the loss of dexterity as the number one problem in cold weather. Dexterity is needed for many important military tasks such as marksmanship, fixing equipment and treating patients. Some specific tasks that are impacted by a loss of dexterity in cold weather include a reduction in weapons accuracy due to “pulling” with the cold fingers vs “squeezing” the trigger, manipulating buttons on communications gear, putting on protective masks and using the emergency release on the MOLLE pack.

Cold exposure decreases manual dexterity through an increase in sympathetic vasoconstriction and a decrease in blood flow, causing a significant fall in skin temperature. Gloves can maintain finger skin temperatures at comfortable levels and protect against cold injury, but dexterity is reduced by 50-80% (see references 2; 6; and 11 at the end of the specification). The resulting impaired hand performance undermines combat and non-combat military operations. Heated gloves, although they can better maintain finger and hand temperatures (ref. 2), do not improve dexterity back to levels observed when bare-handed, due to glove stiffness and materials used. If gloves are not used at all or electrically-heated gloves need to be taken off, hands/fingers cool rapidly, and dexterity is severely impaired as finger temperatures fall below 15° C., likely due to changes in joint mobility and nerve conduction velocity (see refs. 10; 13).

The primary dexterity task impacted by cold exposure is fine motor dexterity. In the civilian world, dexterity is also important for a number of occupations that need optimal hand function in cold conditions, including power-line workers, vehicle maintenance crews, construction workers, fisherman, and recreational athletes (e.g., sailors, climbers, hunters).

Solutions for warfighters and civilian workers are needed that maintain gross and fine-motor dexterity during cold-weather operations equal to that observed during bare-handed exposure in temperate environments. Any solution must be lightweight and if external power is required, only a small amount should be required.

### SUMMARY OF THE INVENTION

One aspect of the invention is an apparatus for improving the dexterity of the bare hands of a human in cold temperatures. The apparatus includes a first heating pad configured for disposal on a forehead of the human, a second heating pad configured for disposal on a first cheek of the human, a third heating pad configured for disposal on a second cheek of the human, a fourth heating pad configured for disposal on one forearm of the human and a fifth heating pad configured for disposal on another forearm of the human. A power supply is connected to each of the heating pads. Thermocouples are disposed on each of the heating pads. An adjustable set-point controller is connected to the thermocouples and to the power supply.

In one embodiment, each of the heating pads includes an outer layer of polyester fleece, a Mylar® layer adjacent the outer layer to reflect heat back to skin of the human, a Kapton® heating element layer adjacent the Mylar® layer and an inner layer of kinesiology tape adjacent the skin of the human

The adjustable set-point controller may include a high/low temperature alert and a high temperature safety limit.

The set-point temperature of each of the heating pads may be about 42° C.

The combined surface area of the first, second and third heating pads is at least about 0.03 square meters. The combined surface area of the fourth and fifth heating pads is at least about 0.117 square meters.

Another aspect of the invention is a method for improving dexterity of bare hands of a human in cold temperatures. The method includes placing a first heating pad on a forehead of the human, placing second and third heating pads on respective cheeks of the human and generating heat in each of the heating pads.

The method may include placing fourth and fifth heating pads on respective forearms of the human.

The method includes measuring a temperature of each heating pad and providing the measured temperatures to an adjustable set-point controller.

The invention will be better understood, and further objects, features and advantages of the invention will become more apparent from the following description, taken in conjunction with the accompanying drawings.

### BRIEF DESCRIPTION OF THE DRAWINGS

In the drawings, which are not necessarily to scale, like or corresponding parts are denoted by like or corresponding reference numerals.

FIG. 1 is a schematic diagram of one embodiment of a heating pad for use with the invention.

FIG. 2A illustrates the placement of heating pads on a human face.

FIG. 2B is a side view of FIG. 2A.

FIG. 3 shows the placement of a heating pad on a forearm.

FIG. 4. is a schematic diagram of one embodiment of a personal heating dexterity device.

FIG. 5 is a graph of hand temperature for each of four trials after 90 minutes of cold exposure.

FIG. 6 is a graph of finger temperature for each of four trials after 90 minutes of cold exposure.

FIGS. 7 and 8 show the change in dexterity after 45 and 90 minutes of cold exposure for each of four trials.

FIGS. 9 and 10 show the change in finger strength after 45 and 90 minutes of cold exposure for each of four trials.

### DETAILED DESCRIPTION

Indirect heating methods have been utilized to maintain peripheral blood flow in the hands and fingers over the last 80 years. These studies support the hypothesis that heating one area causes vasodilation and an increase in skin temperatures in the extremities.

Multiple studies conducted from 1932 to 1947 found that leg immersion in 42-44° C. water caused hand skin temperature to increase from 21° C. to 33° C. (ref. 9) in 14° C. air, to increase hand temperature from 30 to 32° C. in 17° C., (with a fall in hand temperature to 28° C. with no leg heating/control) (ref. 15) and to increase hand blood flow by 400% (ref. 8) during 17° C. air exposure.

Rapaport et al. (ref. 19) provided torso heating during experiments in ambient temperatures that ranged from minus 17 to minus 35° C. They were able to keep the hand temperature above 29° C. when heating was applied from the beginning of cold exposure. In several trials (air temperature of −18° C.), they allowed the hand temperature to fall to 16° C. and then provided torso heating. The hand temperature increased to 32° C.

From 1998-2001, Defence Research and Development Canada (DRDC) published four well designed studies examining the impact of indirect and direct heating on finger and toe temperatures and extending their findings to dexterity. Indirect heating studies were conducted by heating the torso (0.366 m²) to 42° C., which required 108 Watts of power (ref. 3). In the first study, volunteers were tested during a 3 hour exposure to −15° C. air while wearing 2.6 do of clothing insulation. Initially, finger temperature was allowed to drop to 15° C. in bare-handed subjects (took ˜10 minutes). At this point, the torso heater was either turned on or the subject donned gloves. During the control trial (gloves), finger temperature increased from 15° to 18° C. in the first ten minutes upon donning, but then subsequently fell to 12° C. and remained there until the 3 hour experiment was concluded. During torso heating (bare-handed), finger temperature increased to 25° C. over the first hour of exposure and remained there for the duration. Finger blood flow increased ˜500% in the torso heating trial, but did not change in the glove trial. Toe temperature during the glove trial fell to 11° C. after 2.5 hours of exposure, whereas in the torso heating trial, mean toe temperature increased to 22° C. after 90 minutes and remained there. Volunteers reported much greater comfort during the torso heating trials.

In study 2 (ref. 4), the relationship between body heat content and finger temperature/dexterity was studied during exposure to −25° C. air. Eight men completed four trials: a) bare-handed with torso heating and heavy clothing insulation (3.6 do); b) bare-handed, torso heating and less insulation (2.4 clo); c) heavy insulation with heating, contact gloves and mittens, and d) heavy insulation, gloves, mittens, but with no torso heating. These four conditions provided a scenario that resulted in different levels of heat storage. The most important finding was the direct linear relationship between the change in body heat content and finger temperature. Finger temperatures were maintained above 23° C. (comfortable) when the total body heat loss was no more than 250 kilojoules.

In study 3 (ref. 2), finger dexterity was directly studied during exposure to −25° C. air (insulation worn was 3.6 do). Comparisons were made between a heated vest (indirect heating) and heated gloves (direct heating). Relationships were studied between dexterity and blood flow, body heat content, mean skin temperature, and forearm muscle temperature. They found no difference in dexterity between the two heating methods, despite large differences in finger blood flow and body heat content and smaller differences for mean skin temperature and forearm muscle temperature. Maintenance of finger temperature was believed the reason for the lack of differences between trials. One important point from this study was that dexterity was studied with the gloves on. In study 4 (ref. 7), these authors demonstrated that finger blood flow was an important factor for maintaining dexterity when the tests were done bare-handed. Thus finger blood flow may still be the most important variable for maintaining dexterity near levels observed in thermoneutral conditions.

In summary, this series of studies demonstrated that heating, whether delivered indirectly through the torso, or directly to the hand, has the potential to maintain dexterity. Torso heating appears to be the better choice as dexterity could be maintained during extended exposures to cold in bare-handed individuals, such that dexterity is similar to that measured in a temperate environment. In addition, torso heating also improves toe temperatures. The drawback of this heating method is that it requires a large amount of power. In a dismounted soldier, this would mean carrying power supplies that add mass and volume to the soldier, which is not desirable. Heated gloves are problematic in that they are more likely to wear and tear during normal use in cold environments. Plus, as stated before, dexterity is reduced 50-80% when gloves are worn, compared to bare-handed conditions. A solution is needed that will increase finger blood flow, and keep finger temperatures relatively high while the hands are bare, but consume little power.

### Face Heating

Face cooling causes a peripheral vasoconstriction and decreases blood flow to the extremities (refs. 5; 12; 14). This is known as the trigeminal-sympathetic-peripheral vasculature pathway, as stimulation of the trigeminal nerve triggers this response. The trigeminal nerve is composed of three large branches. They are the ophthalmic (V1, sensory), maxillary (V2, sensory) and mandibular (V3, motor and sensory) branches. The most important parts that trigger peripheral vasoconstriction are the areas around the forehead, nose, and eyes because of the high receptor density in these areas. This is likely due to the ophthalmic division of the trigeminal nerve, which is very sensitive to cold (ref. 18). Stimulation of this reflex also causes bradycardia and is known as the diving reflex. Per unit area of skin, facial temperature exerts the largest peripheral influence on autonomic thermoregulation (ref. 16). Thus, the face appears to be an area to exploit for maintaining peripheral blood flow by not allowing it to cool.

Two studies support this supposition. Bader et al. (ref. 1) measured hand/finger temperature during exposure to 15° C. (wearing undershirt, undershorts, trousers, shoes, socks) in three different trials. They were: a) whole face (surface area of 378 cm²) heating (42° C.); b) chest heating (same small surface area heated as the face, 42° C.); and c) leg and foot heating (42° C.). They observed that chest and lower limb heating had no impact on finger temperature, but that face heating caused the finger temperature to rise by 9.8° C. over an 80-minute period.

O'Brien et al. (ref. 17) studied the effect of covering the face with a balaclava on finger temperature during exposure to −15° C. air temperature and a 3 m·s-1 wind. During the first 30 minutes of exposure, finger temperature was 2-4° C. warmer when the face was protected, compared to the uncovered face. Furthermore, wearing a balaclava resulted in a higher mean skin and core temperature. Dexterity was not affected by facial protection. At the first measurement, finger temperatures in both conditions were above the finger temperature that decrements are typically observed (15° C.). At the second dexterity assessment (60 min exposure), finger temperatures in both trials were below 15° C. and thus decrements in dexterity were observed in both trials.

In summary, these two studies support the hypothesis that maintaining or heating the facial region will result in less peripheral vasoconstriction and warmer hands and fingers. Heating the face appears to have a greater potential to impact peripheral temperatures than trying to maintain higher temperatures through passive means (covering the face). Thus facial heating is a candidate for maintaining dexterity during cold exposure.

### Forearm Heating Only

From 2007 through 2010, Phase 1 SBIR grants were given to several companies. The objective was to “develop a lightweight, robust, low-energy, non-flammable, system or method for maintaining fine-motor dexterity of the hands and fingers in resting individuals during cold weather-operations for up to 4 h in ambient temperatures of less than 32° F.” One company developed a prototype that covered the forearm and dorsal aspect of the hand. Their results were promising. Heating the forearm sleeve to 42° C. in three different ambient temperatures (0° C., −7° C., −15° C.), they found that dexterity was better maintained at 0° C. (90% of what was observed in baseline temperate conditions) compared to no heating (60% of baseline). At −7° C. and −15° C., dexterity was maintained at ˜70% of baseline values with heating compared to 40-50% of baseline in the no heating condition. These dexterity differences were achieved using ˜6.7 W of energy, powered by a 12-volt battery.

### Additional Research

Additional research was conducted to determine if focused forearm heating, face heating, or combined forearm and face heating can maintain peripheral skin temperatures and manual dexterity during cold air exposure, compared to no heating. The hypothesis was that hand and finger temperatures would be higher and dexterity performance better when focused heating was provided by combined arm and face heating, compared to no heating or single heating sites.

The eight volunteers for the research were six men and two women with ages of 26±nine years; heights of 170±6 centimeters; weights of 77.6±16.2 kg; and body fat of 16.4±5.3%. A cross-over randomized design was used. The design included no heating (CON); forearm heating only (ARM); face heating only (FACE); and combined forearm and face heating (COMB). Temperature measurements on the subjects included core, hand, finger and eleven other skin sites. Dexterity and strength tests included Purdue Pegboard Assembly; Magazine (ammunition) Loading Task; and Tip, Key and Palmer Pinch Strength. The test subjects were barehanded and clothed with about 2 Clo clothing insulation to maintain stable heat storage. Cold exposure was at 0° C. with 1.34 msec wind.

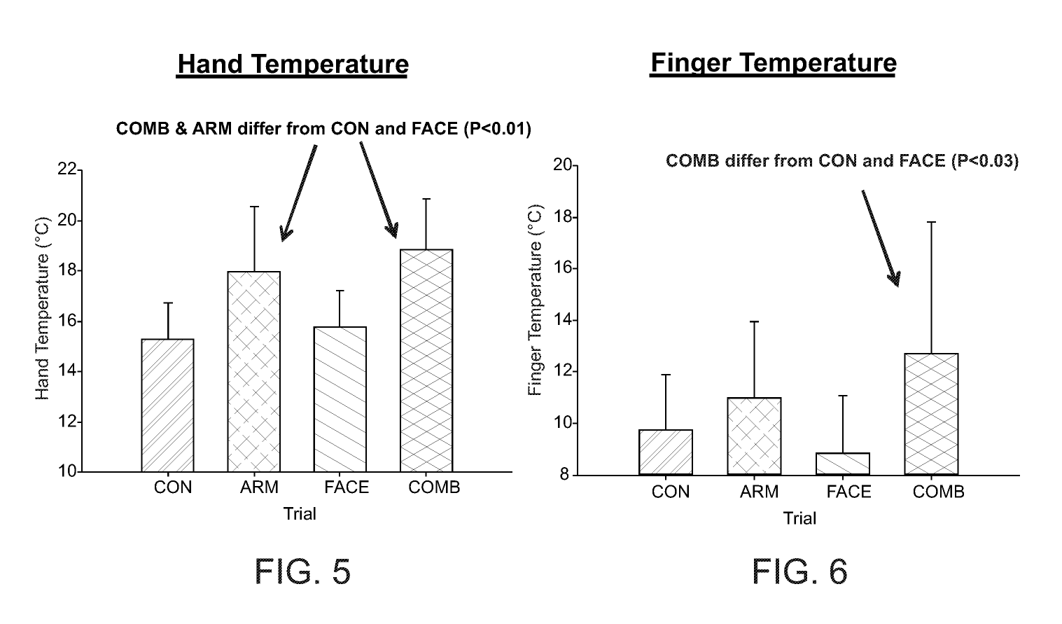

FIGS. 5 and 6 show hand temperature and finger temperature after 90 minutes of cold exposure for each trial, that is, for CON (no heating), ARM (forearm heating only), FACE (face heating only) and COMB (combined forearm and face heating).

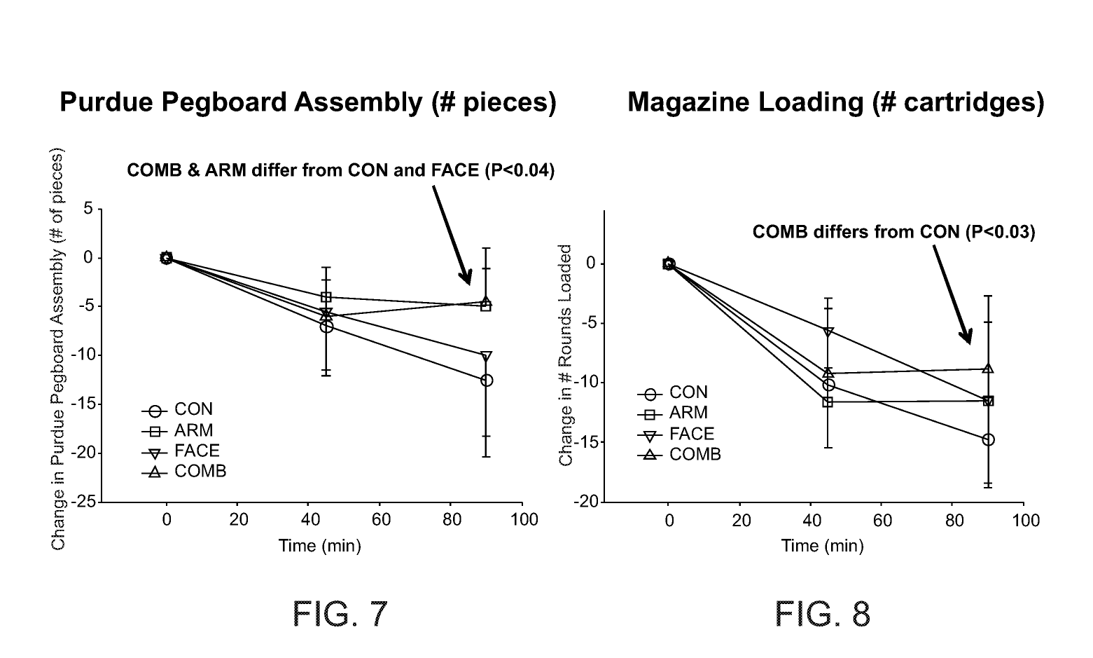

FIGS. 7 and 8 show the change in dexterity measured after 45 and 90 minutes of cold exposure for CON, ARM, FACE and COMB. FIG. 7 shows the results of the Purdue Pegboard Assembly test and FIG. 8 shows the results of the Magazine Loading test. In FIG. 7, after 90 minutes of cold exposure, the percent change for CON is −35±20%; the percent change for ARM is −14±20%; the percent change for FACE is −31±27% ; and the percent change for COMB is −14+10%. In FIG. 8, after 90 minutes of cold exposure, the percent change for CON is −23±5%; the percent change for ARM is −17±9%; the percent change for FACE is −19±13% ; and the percent change for COMB is −14±10%.

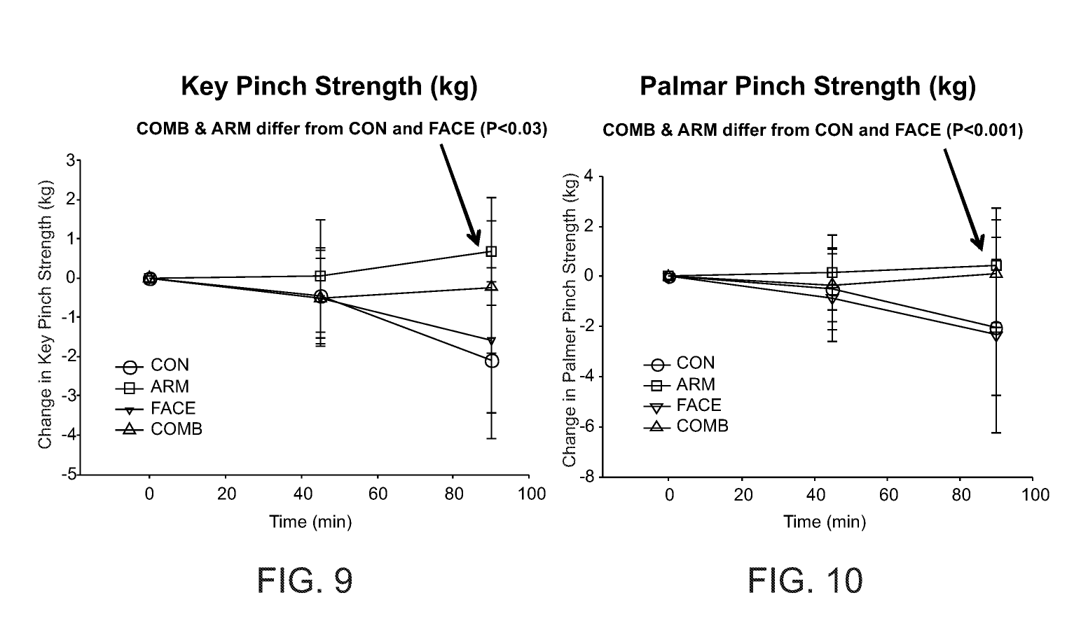

FIGS. 9 and 10 show the change in finger strength measured after 45 and 90 minutes of cold exposure for CON, ARM, FACE and COMB. FIG. 9 shows the results of the Key Pinch Strength test and FIG. 8 shows the results of the Palmar Pinch Strength test. In FIG. 9, after 90 minutes of cold exposure, the percent change for CON is −20±20%; the percent change for ARM is +7±14%; the percent change for FACE is −16±20% ; and the percent change for COMB is −2±16%. In FIG. 10, after 90 minutes of cold exposure, the percent change for CON is −19±24%; the percent change for ARM is +7±21%; the percent change for FACE is −20±36% ; and the percent change for COMB is +3±21%.

Combined arm and face heating (COMB), compared to no heating (CON) and face heating (FACE) heating only, increased hand temperatures by 3.1-3.4° C. and finger temperatures by 3.5-3.9° C. This improved fine and gross manual dexterity by 50%.

Arm heating alone (ARM), compared to CON and FACE, resulted in 2.2-2.5° C. higher hand temperatures and 1.8-2.2° C. higher finger temperatures. This resulted in a 50% improvement in fine motor dexterity.

Finger strength was maintained at baseline levels in ARM and COMB after 90 minutes of cold exposure whereas in CON and FACE, strength decreased by 15-20%.

These data suggest that a combination of forearm and face heating or forearm heating alone is a potential countermeasure for limiting cold-induced dexterity and finger strength losses.

### Exemplary Embodiments

The novel features of the invention include its positive impact on gloveless, gross functional performance and increased fine-motor dexterity achieved by simultaneously heating the face and forearm. Heating the face exploits a physiological reflex and tricks the body into believing it is warm rather than cold, and increases the blood flow to the fingers. By combining facial and forearm heating, the invention gives bare-handed individuals the increased manual dexterity and thermal comfort needed in cold weather environments.

The invention is designed to increase finger blood flow and manual dexterity during cold exposure. It delivers heat specifically to the trigeminal nerve/reflex area of the face (forehead, cheeks) and to the forearms to reduce peripheral vasoconstriction in the hands and fingers that normally occurs with cold exposure.

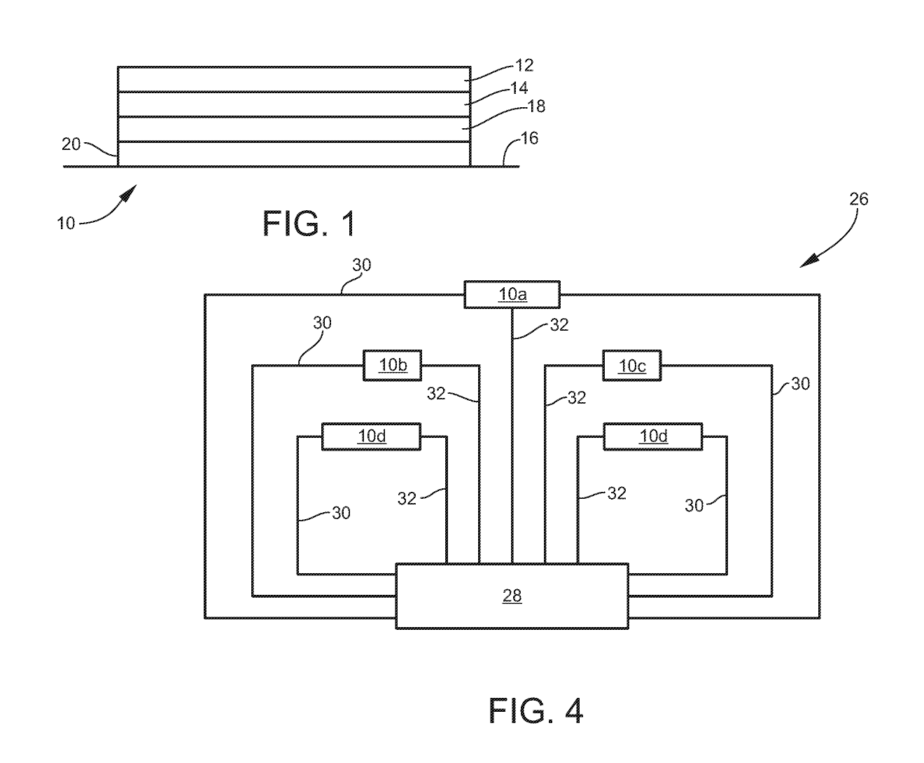

FIG. 1 is a schematic diagram of one embodiment of a heating pad 10 according to one aspect of the invention. The heating pads 10 for the face and forearms have four layers: a) an outer layer 12 of polyester fleece to reduce heat loss to the environment; b) a layer 14 of Mylar® to reflect heat back to the skin 16 and reduce heat loss to the environment; c) the heating element 18, made from Kapton®, a flexible, clear, amber colored polyimide film; and d) an inner layer 20 of kinesiology tape that has direct contact with the skin 16.

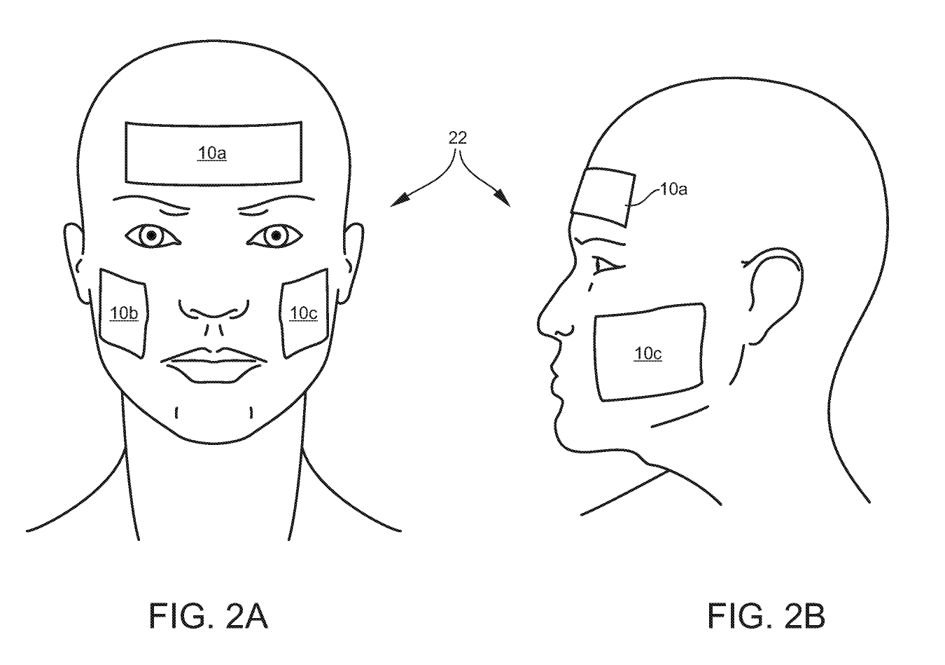

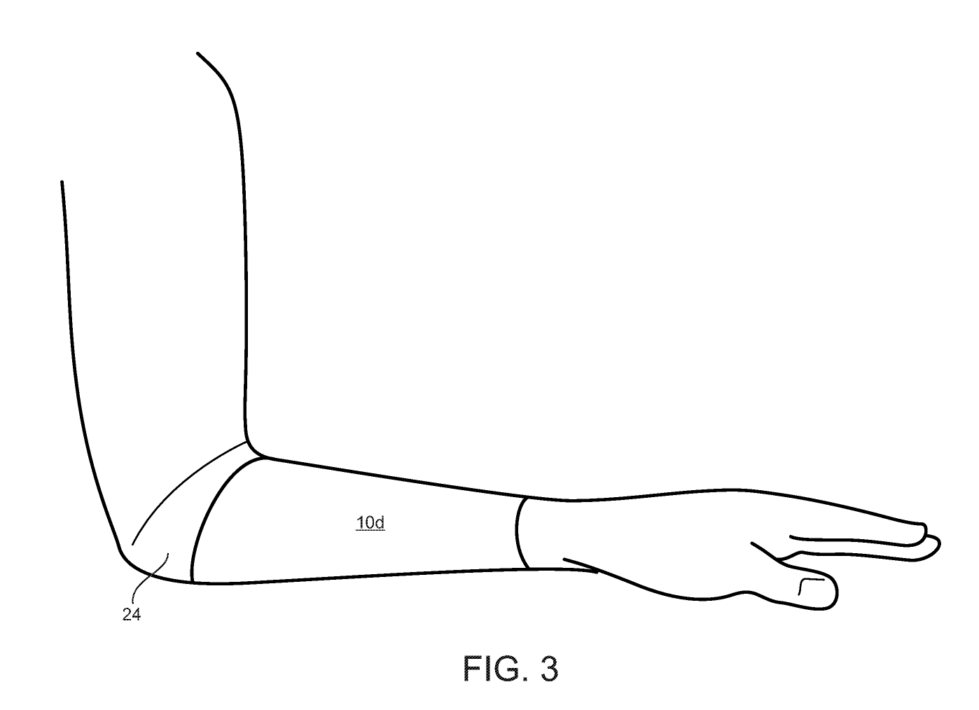

FIGS. 2A and 2B illustrate the placement of heating pads 10 on the human face 22. Pad 10a is a forehead pad and pads 10b and 10c are cheek pads. Facial heating occurs over the forehead and both cheeks. The combined surface area of the forehead pad 10a and cheek pads 10b and 10c is at least about 0.03 m². FIG. 3 shows the placement of a heating pad 10d on a forearm 24. Forearm heating is on both forearms. The combined surface area of the two forearm heating pads 10d, 10d is at least about 0.117 m². The set-point temperature on both the face and forearm pads is 42° C.

FIG. 4. is a schematic diagram of one embodiment of a personal heating dexterity device 26. The Kapton® heating element layer 18 of each heating pad 10a-10d is controlled by a multi-function, adjustable set-point controller 28 equipped with a high/low temperature alert and a high temperature safety limit. The controller 28 may include a battery power supply or the battery power supply may be a separate unit. Power from the controller/power supply 28 is provided to the heating element layer 18 of each heating pad via wiring 30. Thermocouples 32 are connected to the controller 28 from each heating pad. Thermocouples 32 may be located on the surface of the Kapton® layer 18. Thermocouples 32 are used for temperature control and to ensure safe temperature limits. Kapton® heaters have been safety tested and approved, and they are UL recognized under the UL file number E251285.

The apparatus 26 may be incorporated into one or more garments. For example, the forehead heating pad 10a and cheek heating pads 10b, 10c may be incorporated into a balaclava type of cloth headgear. The forearm heating pads 10d may be incorporated into an upper body garment such as a shirt or blouse. The controller 28 may be disposed in the pocket of a garment or worn on a belt.

Embodiments of the invention have been described to explain the nature of the invention. Those skilled in the art may make changes in the details, materials, steps and arrangement of the described embodiments within the principle and scope of the invention, as expressed in the appended claims.

### REFERENCE LIST

1. Bader M E and Macht M B. Indirect peripheral vasodilatation produced by the warming of various body areas. J Appl Physiol 1: 215-226, 1948.

2. Brajkovic D and Ducharme M B. Finger dexterity, skin temperature, and blood flow during auxiliary heating in the cold. J Appl Physiol 95: 758-770, 2003.

3. Brajkovic D, Ducharme M B and Frim J. Influence of localized auxiliary heating on hand comfort during cold exposure. J Appl Physiol 85: 2054-2065, 1998.

4. Brajkovic D, Ducharme M B and Frim J. Relationship between body heat content and finger temperature during cold exposure. J Appl Physiol 90: 2445-2452, 2001.

5. Brown C M, Sanya E O and Hilz M J. Effect of cold face stimulation on cerebral blood flow in humans. Brain Res Bull 61: 81-86, 2003.

6. Dianat I, Haslegrave C M and Stedmon A W. Methodology for evaluating gloves in relation to the effects on hand performance capabilities: a literature review. Ergonomics 55: 1429-1451, 2012.

7. Ducharme M B, Brajkovic D and Frim J. The effect of direct and indirect hand heating on finger blood flow and dexterity during cold exposure. J Therm Biol 391-396, 1999.

8. Ferris B G, Forster R E, II, Pillion E L and Christensen W R. Control of peripheral blood flow: repsonses in the human hand when extremities are warmed. Am J Physiol 304-314, 1947.

9. Gibbon J H and Landis E M. Vasodilatation in the lower extremities in response to immersing the forearm in warm water. J Clin Invest 11: 1019-1036, 1932.

10. Havenith G, Heus R and Daanen H A M. The hand in the cold, performance and risk. Arctic Med Res 37-47, 1995.

11. Havenith G and Vrijkotte T G M. Effectiveness of personal protective equipment for skin protection while working with pesticides in greenhouses. In: Part III: Comfort and Ergonomics, Soesterberg: TNO Human Factors Research Institute: Report No. IZF-C40, 1993.

12. Heath M E and Downey J A. The cold face test (diving reflex) in clinical autonomic assessment: methodological considerations and repeatability of responses. Clin Sci 78: 139-147, 1990.

13. Heus R, Daanen H A M and Havenith G. Physiological criteria for functioning of hands in the cold. Appl Ergonomics 26: 5-13, 1995.

14. Hilz M J, Stemper B, Sauer P, Haertl U, Singer W and Axelrod FB. Cold face test demonstrates parasympathetic cardiac dysfunction in familial dysautonomia. Am J Physiol 1833-1839, 1999.

15. Martinez C and Visscher M B. Some observations on general skin temperature responses to local heating of human subjects in a cold environment. Am J Physiol 724-734, 1945.

16. Nakamura M, Yoda T, Crawshaw L I, Yasuhara S, Saito Y, Kasuga M, Nagashima K and Kanosue K. Regional differences in temperature sensation and thermal comfort in humans. J Appl Physiol 1897-1906, 2008.

17. O'Brien C, Castellani J W and Sawka M N. Thermal face protection delays finger cooling and improves thermal comfort during cold air exposure. Eur J Appl Physiol 111: 3097-3105, 2011.

18. Parfrey P and Sheehan J D. Individual facial areas in the human circulatory response to immersion. Irish J Med Sci 144: 335-342, 1975.

19. Rapaport S I, Fetcher E S, Shaub H G and Hall J F. Control of blood flow to the extremities at low ambient temperatures. J Appl Physiol 2: 61-71, 1949.

What is claimed is:

1. An apparatus for improving dexterity of bare hands of a human in cold temperatures, comprising:
a first heating pad configured for disposal on a forehead of the human;
a second heating pad configured for disposal on a first cheek of the human;
a third heating pad configured for disposal on a second cheek of the human;
a fourth heating pad configured for disposal on one forearm of the human;
a fifth heating pad configured for disposal on another forearm of the human;
a power supply connected to each of the heating pads;
thermocouples disposed on each of the heating pads; and
an adjustable set-point controller connected to the thermocouples and to the power supply.

2. The apparatus of claim 1, wherein each of the heating pads includes an outer layer of polyester fleece, a Mylar® layer adjacent the outer layer to reflect heat back to skin of the human, a Kapton® heating element layer adjacent the Mylar® layer and an inner layer of kinesiology tape adjacent the skin of the human

3. The apparatus of claim 1, wherein the adjustable set-point controller includes a high/low temperature alert and a high temperature safety limit.

4. The apparatus of claim 3, wherein a set-point temperature of each of the heating pads is about 42° C.

5. The apparatus of claim 4, wherein a combined surface area of the first, second and third heating pads is at least about 0.03 square meters.

6. The apparatus of claim 5, wherein a combined surface area of the fourth and fifth heating pads is at least about 0.117 square meters.

7. A method for improving dexterity of bare hands of a human in cold temperatures, comprising:
placing a first heating pad on a forehead of the human;
placing second and third heating pads on respective cheeks of the human; and
generating heat in each of the heating pads.

8. The method of claim 7, further comprising placing fourth and fifth heating pads on respective forearms of the human.

9. The method of claim 8, further comprising measuring a temperature of each heating pad and providing the measured temperatures to an adjustable set-point controller.

10. The method of claim 8, wherein the steps of placing heating pads includes placing heating pads made of an outer layer of polyester fleece, a Mylar® layer adjacent the outer layer to reflect heat back to skin of the human, a Kapton® heating element layer adjacent the Mylar® layer and an inner layer of kinesiology tape adjacent the skin of the human.

11. The method of claim 9, wherein the step of generating heat includes generating heat in a heating pad until a temperature of the heating pad is about 42° C.
